(19) United States
(12) Patent Application Publication
(10) Pub. No.: US 2017/0157098 A1
(43) Pub. Date: Jun. 8, 2017

(54) Clopidogrel Hydrogen Sulfate Solid Preparation and Its Preparation Method

(71) Applicants: SHENZHEN SALUBRIS PHARMACEUTICALS CO., LTD, Shenzhen (CN); SHANDONG SALUBRIS PHARMACEUTICALS CO., LTD, Dezhou (CN); HUIZHOU SALUBRIS PHARMACEUTICALS CO., LTD., Huizhou (CN)

(72) Inventors: Yanxia CAI, Shenzhen (CN); Yueyao TAN, Shenzhen (CN); Guanhao YE, Shenzhen (CN)

(21) Appl. No.: 15/284,218

(22) Filed: Oct. 3, 2016

(30) Foreign Application Priority Data
Dec. 4, 2015 (CN) 201510888830.7

(51) Int. Cl. A61K 31/4365; A61K 9/20

(52) U.S. Cl. CPC A61K 31/4365; A61K 9/2031; A61K 9/2054; A61K 9/2027; A61K 9/2018; A61K 9/2095

(57) ABSTRACT

To provide a Clopidogrel Hydrogen Sulfate solid preparation, melting granulation process is used, the content of raw material in the granules obtained is high, and the preparation is of good homogeneity, smooth surface, small tablet weight variation and dissolution complying with the clinical medication. When specific crystal form is used, the effect is better.

This application claims priority from a Chinese utility patent application, application number 201510888830.7, Clopidogrel Hydrogen Sulfate Solid Preparation and Its Preparation Method, filed on 4 Dec. 2015, which is incorporated herein in its entirety by reference.

### TECHNOLOGY FIELD

This invention belongs to the field of pharmaceutical preparation, which specially involves in a Clopidogrel Hydrogen Sulfate solid preparation and its preparation method.

### BACKGROUND TECHNOLOGY

Clopidogrel Hydrogen Sulfate (CAS: 135046-48-9) is the sulfate of clopidogrel. Its English name is Clopidogrel Hydrogen Sulfate and chemical name is (s)-α-(2-Chlorophenyl)-6,7-dihydrothieno [3,2-c] pyridine-5(4H) acetate hydrogen sulfate. As an anti-platelet aggregation agent, Clopidogrel Hydrogen Sulfate is developed by Sanofi-Aventis in France and launched Britain and America firstly in 1998. Clopidogrel Hydrogen Sulfate launched China in 2001, and is clinically used for the prevention of atherosclerosis and thrombosis. At present, Clopidogrel Hydrogen Sulfate preparations in China mainly are Plavix manufactured by Sanofi-aventis and Talcom manufactured by Shenzhen Salubris Pharmaceutical Co., Ltd.

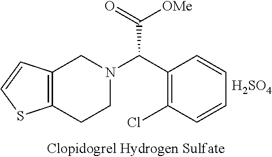

Clopidogrel Hydrogen Sulfate is unstable in damp and hot environment. Therefore, the common wet granulation-tabletting process is not applicable to Clopidogrel Hydrogen Sulfate preparation. Currently dry process is commonly used in preparation of Clopidogrel Hydrogen Sulfate. However, as Clopidogrel Hydrogen Sulfate raw material is hygroscopic and its powder electrostatic phenomenon is serious, if direct compressing process is used to prepare the solid preparation, on one hand, it is not suitable for direct compression due to poor mobility of total blending powder, and on the other hand, sticking may occur during compressing to make manufacturing unsuccessful due to compound properties and/or formula, Therefore, to prepare Clopidogrel Hydrogen Sulfate solid preparation, granulating procedure is usually required to prepare active ingredient power with poor mobility and wide particle size distribution into granule applicable to compressing. The current technologies mainly are dry granulation and melting granulation.

Patent CN1935119A discloses a dry granulation process for the preparation of Clopidogrel Hydrogen Sulfate solid preparation, and the solid preparation obtained shows high stability. However, serious powder electrostatic phenomenon makes poor mixing property during mixing and poor homogeneity of the active ingredient. Qualified preparation was prepared by methods such as formula improvement, but the preparation process is not the most optimal one as a whole.

During the melting granulation process, it always uses the excipients with low melting points as binder, and the granulation is formed by melting and cooling following by gathering and adhesion. This method can improve effectively the homogeneity of raw material and excipients in the granulation, however, for the melting granulation of Clopidogrel Hydrogen Sulfate, its active ingredient is of property easy to sticking, which makes the granulation obtained meeting the requirement of the follow-up compression be ensured during the development of the melting granulation process. Patent CN101690719 discloses a series of Clopidogrel Hydrogen Sulfate solid preparations, which use PEG6000 as binder and are prepared by melting granulation at 50˜90° C. in jacketed kettle or fluidized bed. The process improves the mixing uniformity of raw material and excipients and relieves sticking effectively and the solid preparations obtained are of high stability. However, in this melting granulation process, more excipients per batch is used, that is, Clopidogrel Hydrogen Sulfate per unit mass requires more excipients to disperse. It is concluded that this process also remains to be improved.

Chinese Journal of Pharmaceuticals (2011, Volume 46, No. 2, page 117) discloses a melting granulation process of Clopidogrel Hydrogen Sulfate, which uses PEG6000 as binder and uses the melting granulation process. This process solves sticking effectively, the friability and tablet weight variation of solid preparation obtained is less, and dissolution complies with the requirements of clinical medication. The literature, whereafter, states the effect of melting binder usage on granulation, which considers that too little binder makes it difficult to prepare granule, and too much binder makes it easy to produce hard granule with block mass, thus affecting the dissolution of preparation. However, excipient used in each batch of this melting granulation process are comparatively too much, and the process remains to be improved.

Consequently, to reduce materials used in the batches of melting granulation, to improve the content of raw materials in granulation, to optimize the preparation process on the whole and to finally prepare Clopidogrel Hydrogen Sulfate solid preparation complying with the requirements of clinical medication are issues not have been solved by current technologies.

### INTRODUCTION TO THE INVENTION

The invention aims to overcome the deficiency of the current technologies and provides a Clopidogrel Hydrogen Sulfate solid preparation, which uses melting granulation process to prepare granulation with high content of raw material, and preparation obtained with characteristics of good homogeneity, smooth surface, small tablet weight variation, and dissolution complying with the requirements of clinical medication.

Purposes of the invention mentioned above are achieved by the following technological methods:

A Clopidogrel Hydrogen Sulfate solid preparation contains granule and extragranular excipients, of which the granule contains Clopidogrel Hydrogen Sulfate, filler, disintegrant and binder. The Clopidogrel Hydrogen Sulfate solid preparation mentioned uses melting granulation process, and its characteristics are that D90 of Clopidogrel Hydrogen Sulfate mentioned is 30˜100 μm and binder is PEG6000. When the mass ratio of Clopidogrel Hydrogen Sulfate is 1, the usage of binder is 0.1˜0.5.

The key of invention is to control grain diameter (D90) of Clopidogrel Hydrogen Sulfate and usage of binder PEG6000. If no special instructions, Clopidogrel Hydrogen Sulfate mentioned in this invention is calculated on Clopidogrel Hydrogen Sulfate.

In current melt granulation process disclosed, to ensure granulation effect and dissolution property, a lot of binder and then excipients are needed, which ensure Clopidogrel Hydrogen Sulfate dispersed as uniformly as possible in the granule, but affect preparation efficiency. To improve preparation efficiency, we performed a large number of experiments to increase the content of Clopidogrel Hydrogen Sulfate in granule. In the research and development, we found surprisingly that, when the granule diameter (D90) of Clopidogrel Hydrogen Sulfate is controlled between 30 and 100 μm, the usage of binder PEG6000 can be reduced effectively, and thus the usage amount of excipients was reduced consequently. The diameter of the granule obtained is homogeneous and granule shows good mobility. The granule is easy to mix with extragranular excipients and shows no sticking phenomenon in the follow-up compressing. The tablets obtained are of good homogeneity, smooth surface, small tablet weight variation and dissolution complying with the requirements of clinical medication. More specifically, the more usage amount of binder in certain range, the more excipients will be used, which is more in favor of obtaining qualified granules. Increase in the granule diameter of Clopidogrel Hydrogen Sulfate can reduce the usage amount of binder to a certain extent, however, when granule diameter increases to a certain degree, there is no decrease in the usage amount of binder any more. In addition, large granule diameter of Clopidogrel Hydrogen Sulfate contributes to obtain large granule diameter of granulation, and also has effect on dissolution rate. Too few usage of binder also affects granulation results, makes the granule loose and easy to break into powders, which goes against the follow-up process; While too small granule diameter of Clopidogrel Hydrogen Sulfate requires more binder, more excipients, and leads small granule diameter of disperse phase granules, which contain more powder as well, and also not in favor of the follow-up process; For the melting granulation process, when the granule diameter (D90) of Clopidogrel Hydrogen Sulfate is controlled between 30 and 100 μm, the optimal quantity of binder PEG6000 possible to be used accordingly, which effectively improves content of Clopidogrel Hydrogen Sulfate in granule, the granule diameter is moderate, and granule distribution is narrow, they all contribute to the optimization of mixing and compressing, and all indicators of tablets obtained comply with the requirements of clinical medication for Clopidogrel Hydrogen Sulfate. binder (D90) of Clopidogrel Hydrogen Sulfate is 40˜90 μm and most preferred 50˜80 μm. Clopidogrel Hydrogen Sulfate solid preparation mentioned in the invention can use Current Clopidogrel Hydrogen Sulfate crystal form, such as crystal form I and II reported. Crystal form I reported in the current technology is preferred, that is, X-ray powder diffraction pattern of the crystal form shows diffraction peaks while 2θ is 9.28°, 10.96°, 14.88°, 15.55°, 18.54°, 19.02°, 20.65°, 23.24°, 23.92° or 25.59°.

On basis of controlling the granule diameter of Clopidogrel Hydrogen Sulfate in the range mentioned above, the usage amount of binder PEG6000 can be reduced, and the usage amount of other excipients is controlled in an appropriate range, thus to improve the content of Clopidogrel Hydrogen Sulfate in granule, and to ensure the granulation effect and to improve granulation efficiency. Specifically, when the mass ratio of Clopidogrel Hydrogen Sulfate is 1, the mass ratio of binder is 0.1˜0.5; and more preferred, when the mass ratio of Clopidogrel Hydrogen Sulfate is 1, the mass ratio of adhesion agent is 0.15˜0.35.

The excipients used in the granule of Clopidogrel Hydrogen Sulfate solid preparation mentioned in the invention is much fewer, and the content of Clopidogrel Hydrogen Sulfate is much higher, which improves effectively the efficiency of melting granulation. The mentioned granule also includes filler and disintegrating agent.

Filler mentioned in granule is one or mixture in various amounts of more than two of microcrystalline cellulose, lactose, pregelatinized starch, etc. It is more preferred for one or mixture in various amounts of more than two of microcrystalline cellulose and lactose. Specifically, when the mass ratio of Clopidogrel Hydrogen Sulfate is 1, the mass ratio of filler is 0.03˜0.1.

The disintegrating agent mentioned in disperse phase granule is one or mixture in various amounts of more than two of polyvinylpolypyrrolidone, low substituted hydroxypropyl cellulose, croscarmellose sodium, sodium carboxymethyl starch, etc. Specifically, when the mass ratio of Clopidogrel Hydrogen Sulfate is 1, the mass ratio of disintegrating agent is 0.2˜0.5.

In one embodiment, the granule formula of this invention is as below:

| Item | Mass Ratio |
|---|---|
| Clopidogrel Hydrogen Sulfate | 1 |
| Microcrystalline cellulose | 0.05 |
| PEG6000 | 0.2 |
| Low substituted hydroxypropyl | 0.3 |
| cellulose | |
| Polyvinylpolypyrrolidone | 0.1 |

In one embodiment, the granule formula of this invention is as below:

| Item | Mass Ratio |
|---|---|
| Clopidogrel Hydrogen Sulfate | 1 |
| Lactose | 0.06 |
| PEG6000 | 0.3 |
| Croscarmellose sodium | 0.3 |

In one embodiment the granule formula of this invention is as below:

| Item | Mass Ratio |
|---|---|
| Clopidogrel Hydrogen Sulfate | 1 |
| Microcrystalline cellulose | 0.05 |
| PEG6000 | 0.2 |
| Polyvinylpolypyrrolidone | 0.4 |

In one embodiment, the granule formula of this invention is as below:

| Item | Mass Ratio |
|---|---|
| Clopidogrel Hydrogen Sulfate | 1 |
| Microcrystalline cellulose | 0.05 |
| PEG6000 | 0.15 |
| Low substituted hydroxypropyl | 0.3 |
| cellulose | |
| Polyvinylpolypyrrolidone | 0.1 |

In one embodiment, the granule formula of this invention is as below:

| Item | Mass Ratio |
|---|---|
| Clopidogrel Hydrogen Sulfate | 1 |
| Lactose | 0.1 |
| PEG6000 | 0.35 |
| Croscarmellose sodium | 0.3 |

The melting granulation mentioned can use the equipment commonly used in current technology, and jacketed kettle or fluidized bed granulating machine is preferred. The material temperature in granulation process is controlled between 50 and 80° C. The diameter of the granules obtained are homogeneous, and there is hardly or little powder. The granule is of high homogeneity active ingredient and of good mobility.

The Clopidogrel Hydrogen Sulfate solid preparation mentioned in this invention also includes extragranular excipients, and extragranular excipients mentioned include filler, disintegrating agent, stabilizer and lubricant.

Specifically, the filler mentioned is one or mixture in various amounts of more than two of microcrystalline cellulose, lactose and pregelatinized starch, etc. Specifically, when mass ratio of Clopidogrel Hydrogen Sulfate is 1, the mass ratio of filler is 0.3˜1.0.

The disintegrating agent mentioned is one or mixture in various amounts of more than two of polyvinylpolypyrrolidone, hydroxypropyl cellulose, low substituted hydroxyprepyl cellulose, croscarmellose sodium, sodium carboxymethyl starch, etc. Specifically, when mass ratio of Clopidogrel Hydrogen Sulfate is 1, the mass ratio of disintegrating agent is 0.1˜0.5.

The stabilizer mentioned is sucrose stearate. Specifically, when mass ratio of Clopidogrel Hydrogen Sulfate is 1, the mass ratio of disintegrating agent is 0.02˜0.08.

The lubricant mentioned is one or mixture in various amounts of more than two of magnesium stearate, hydrogenated castor oil, talcum powder, etc. Specifically, when mass ratio of Clopidogrel Hydrogen Sulfate is 1, the mass ratio of lubricant is 0.03˜1.0.

In one embodiment of this invention, Clopidogrel Hydrogen Sulfate solid preparation formula is as below:

| Item | | Mass Ratio |
|---|---|---|
| Intragranular | Clopidogrel Hydrogen Sulfate | 1 |
| Excipients | Microcrystalline cellulose | 0.05 |
| | PEG6000 | 0.2 |
| | Low substituted hydroxypropyl | 0.3 |
| | cellulose | |
| | Polyvinylpolypyrrolidone | 0.1 |
| Extragranular | Microcrystalline cellulose | 0.5 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

In one embodiment of this invention, Clopidogrel Hydrogen Sulfate solid preparation formula is as below:

| Item | | Mass Ratio |
|---|---|---|
| Intragranular | Clopidogrel Hydrogen Sulfate | 1 |
| Excipients | Lactose | 0.06 |
| | PEG6000 | 0.3 |
| | Croscarmellose sodium | 0.3 |
| Extragranular | Microcrystalline cellulose | 0.5 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

In one embodiment of this invention, Clopidogrel Hydrogen Sulfate solid preparation formula is as below:

| Item | | Mass Ratio |
|---|---|---|
| Intragranular | Clopidogrel Hydrogen Sulfate | 1 |
| Excipients | Microcrystalline cellulose | 0.05 |
| | PEG6000 | 0.2 |
| | Polyvinylpolypyrrolidone | 0.4 |
| Extragranular | Microcrystalline cellulose | 0.5 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

In one embodiment of this invention, Clopidogrel Hydrogen Sulfate solid preparation formula is as below:

| Item | | Mass Ratio |
|---|---|---|
| Intragranular | Clopidogrel Hydrogen Sulfate | 1 |
| Excipients | Microcrystalline cellulose | 0.05 |
| | PEG6000 | 0.15 |
| | Low substituted hydroxypropyl | 0.3 |
| | cellulose | |
| | Polyvinylpolypyrrolidone | 0.1 |
| Extragranular | Microcrystalline cellulose | 0.55 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

In one embodiment of this invention, Clopidogrel Hydrogen Sulfate solid preparation formula is as below:

| Item | | Mass Ratio |
|---|---|---|
| Intragranular | Clopidogrel Hydrogen Sulfate | 1 |
| Excipients | Lactose | 0.1 |
| | PEG6000 | 0.35 |
| | Croscarmellose sodium | 0.3 |
| Extragranular | Microcrystalline cellulose | 0.5 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

Another purpose of this invention is to provide a preparation process of Clopidogrel Hydrogen Sulfate solid preparation mentioned in this invention. This preparation process goes with formula property well, is in favor of giving play to formula advantage, achieves the purpose of improving the content of Clopidogrel Hydrogen Sulfate in granule, and realizes the optimization of preparation process in a further step and finally obtains the Clopidogrel Hydrogen Sulfate solid preparation of high pass percent and complying with clinical medication. And the preparation process mentioned includes the following steps:
1) Mix prescription amount of Clopidogrel Hydrogen Sulfate and intragranular excipients for 20˜30 min, and get mixing powder;
2) Add the mixing powder obtained in step 1) into melting granulation equipment, granulate at 50˜80° C. for 10˜30 min to get granules;

The category, property and usage amount of raw material and excipients, and category of the equipment used in the step mentioned above are all in accordance with those mentioned in the former purpose; in step 1), the common mixing equipment can be used, and the Column Mixing Machine is preferred. Granulation time in step 2) should be controlled between 10 and 30 min, which helpful for preparing the granules complying with the follow-up preparation process.

More specifically, the preparation method mentioned includes steps as below:
1) Mix prescription amount of Clopidogrel Hydrogen Sulfate and intragranular excipients for 20˜30 min, and get mixing powder;
2) Add the mixing powder obtained in step 1) into melting granulation equipment, granulate at 50˜80° C. for 10˜30 min to get granules;
3) Mix the granules obtained in step 2) and prescription amount of extragranular excipients for 10˜20 min to get total mixing granules;
4) Compress the total mixing granules obtained in step 3) to get Clopidogrel Hydrogen Sulfate solid preparation.

The category, property and usage amount of raw material and excipients, and category of the equipment used in the step mentioned above are all in accordance with those mentioned in the former purpose; in step 1) and 3), the common mixing equipment can be used, and Column Mixing Machine is preferred; Granulation time in step 2) should be controlled between 10 and 30 min, which is helpful for preparing the disperse phase granules complying with the follow-up preparation process. Granule diameter of disperse phase granules obtained in step 2) is moderate and granules are homogeneous and hardly have or have little powder, which makes the total mixing granules mentioned in step 3) mix rapidly in short time with extragranular excipients and the mixing effect is good; The melting granulation equipment mentioned in step 2) is jacketed kettle or fluidized bed granulating machine; The preferred compress pressure in step 4) is 3˜5 kgf. Based on the benefits of granule obtained, no sticking occurs during compressing.

Further coating step can be conducted to the Clopidogrel Hydrogen Sulfate solid preparation mentioned. The common coating process can be used, and the coating materials are common purchased or self-made ones, such as opadry, hydroxypropyl methylcellulose, etc.

The invention has the following prominent advantages and benefits compared with the current technology:
1. Provide a Clopidogrel Hydrogen Sulfate solid preparation prepared by melting granulation process. The content of raw material in the granules obtained is high, and the preparation obtained is of good homogeneity, smooth surface and small tablet weight variation, and dissolution complying with the requirements of clinical medication, especially when specific crystal form is used, the effect is better;
2. Provide a preparation process of Clopidogrel Hydrogen Sulfate solid preparation mentioned in the invention that conforms to the formula characteristics well is in favor of giving play to formula advantages, realize the purpose of improving the content of Clopidogrel Hydrogen Sulfate in granules, further achieve the optimization of preparation process, and finally prepare the Clopidogrel Hydrogen Sulfate solid preparation with high pass percent and complying with clinical medication.

### Specific Mode of Execution

Conduct particular description to the invention combined with examples, but the mode of execution of the invention is not limited to this.

In the execution example, use BT-1001 intelligent powder characteristic test equipment to measure angle of repose of the granules of Clopidogrel Hydrogen Sulfate;

In the execution examples, Clopidogrel Hydrogen Sulfate crystal I reported in the current technology is used.

### EXAMPLE 1

### Formula

| Item | | Mass Ratio |
|---|---|---|
| Intragranular | Clopidogrel Hydrogen | 1 |
| Excipients | Sulfate | |
| | Microcrystalline cellulose | 0.05 |
| | PEG6000 | 0.2 |
| | Low substituted | 0.3 |
| | hydroxypropyl cellulose | |
| | Polyvinylpolypyrrolidone | 0.1 |
| Extragranular | Microcrystalline cellulose | 0.5 |
| Excipients | Low substituted | 0.1 |
| | hydroxypropyl cellulose | |
| | Polyvinylpolypyrrolidone | 0.25 |
| | Sucrose stearate | 0.05 |
| | Magnesium stearate | 0.015 |

### Preparation Steps:

1) Mixed the prescription amount of Clopidogrel Hydrogen Sulfate (D90=67 μm) and intragranular excipients for 20˜30 min to get mixing powder;
2) Added the mixing powder obtained in step 1) into fluidized bed granulating machine, granulated at 50˜60° C. for 10˜30 min to get granules (Angle of repose 37°), and the granules obtained were homogeneous and hardly had powder;
3) Mixed the granules obtained in step 2) and prescription amount of extragranular excipients for 10˜20 min to get total mixing granules;
4) Compressed the total mixing granules obtained in step 3) to prepare Clopidogrel Hydrogen Sulfate solid preparation (Strength 75 mg).

No sticking occurred during compressing, and the surface of Clopidogrel Hydrogen Sulfate solid preparation obtained was smooth.

### EXAMPLE 2

### Formula

| Item | | Mass Ratio |
|---|---|---|
| Intragranular | Clopidogrel Hydrogen Sulfate | 1 |
| Excipients | Lactose | 0.06 |
| | PEG6000 | 0.3 |
| | Croscarmellose sodium | 0.3 |
| Extragranular | Microcrystalline cellulose | 0.5 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

### Preparation Steps:

1) Mixed prescription amount of Clopidogrel Hydrogen Sulfate (D90=60 μm) and intragranular excipients for 20˜30 min to get mixing powder;
2) Added the mixing powder obtained in step 1) into fluidized bed granulation machine, granulated at 50˜60° C. for 10˜30 min to get granules (Angle of repose 38°). The granules obtained were homogeneous and hardly had powder;
3) Mixed the granules obtained in step 2) and prescription amount of extragranular excipients for 10˜20 min to get total mixing granules;
4) Compressed the total mixing granules obtained in step 3) to prepare Clopidogrel Hydrogen Sulfate solid preparation (Strength 75 mg).

No sticking occurred during compressing, and the surface of Clopidogrel Hydrogen Sulfate solid preparation obtained was smooth.

### EXAMPLE 3

### Formula

| Item | | Mass Ratio |
|---|---|---|
| Intragranular | Clopidogrel Hydrogen Sulfate | 1 |
| Excipients | Microcrystalline cellulose | 0.05 |
| | PEG6000 | 0.2 |
| | Polyvinylpolypyrrolidone | 0.4 |
| Extragranular | Microcrystalline cellulose | 0.5 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

### Preparation Steps:

1) Mixed the prescription amount of Clopidogrel Hydrogen Sulfate (D90=73 μm) and intragranular excipients for 20˜30 min to get mixing powder;
2) Added the disperse phase of mixing powder obtained in step 1) into fluidized bed granulation machine, granulated at 50˜60° C. for 10˜30 min to get disperse phase of granules (Angle of repose 38°). The granules obtained were homogeneous and hardly had powder;
3) Mixed the granules obtained in step 2) and extragranular excipients for 10˜20 min to get total mixing granules;
4) Compressed the total mixing granules obtained in step 3) to prepare Clopidogrel Hydrogen Sulfate solid preparation (Strength: 75 mg).

No sticking occurred during compressing, and the surface of Clopidogrel Hydrogen Sulfate solid preparation obtained was smooth.

### EXAMPLE 4

### Formula

| Item | | Mass Ratio |
|---|---|---|
| Intragranular | Clopidogrel Hydrogen | 1 |
| Excipients | Sulfate | |
| | Microcrystalline cellulose | 0.05 |
| | PEG6000 | 0.15 |
| | Low substituted | 0.3 |
| | hydroxypropyl cellulose | |
| | Polyvinylpolypyrrolidone | 0.1 |
| Extragranular | Microcrystalline cellulose | 0.55 |
| Excipients | Low substituted | 0.1 |
| | hydroxypropyl cellulose | |
| | Polyvinylpolypyrrolidone | 0.5 |
| | Sucrose stearate | 0.05 |
| | Magnesium stearate | 0.015 |

### Preparation Steps:

1) Mixed the prescription amount of Clopidogrel Hydrogen Sulfate (D90=91 μm) and intragranular excipients for 20˜30 min to get mixing powder;
2) Added the mixing powder obtained in step 1) into fluidized bed granulation machine, granulated at 50˜60° C. for 10˜30 min, and get disperse phase of granules (angle of repose 40°).
   The granules obtained were homogeneous and had little powder, but had no effect on the follow-up process;
3) Mixed the granules obtained in step 2) and extragranular excipients for 10˜20 min to get total mixing granules;
4) Compressed the total mixing granules obtained in step 3) to prepare Clopidogrel Hydrogen Sulfate solid preparation (Strength: 75 mg).

No sticking occurred during compressing, and the surface of Clopidogrel Hydrogen Sulfate solid preparation obtained was smooth.

### EXAMPLE 5

### Formula

| Item | | Mass Ratio |
|---|---|---|
| Intragranular | Clopidogrel Hydrogen Sulfate | 1 |
| Excipients | Lactose | 0.1 |
| | PEG6000 | 0.35 |
| | Croscarmellose sodium | 0.3 |
| Extragranular | Microcrystalline cellulose | 0.5 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

### Preparation Steps:

1) Mixed the prescription amount of Clopidogrel Hydrogen Sulfate (D90=48 μm) and excipients for 20˜30 min to get mixing powder;
2) Added the mixing powder obtained in step 1) into fluidized bed granulation machine, granulated at 50˜60° C. for 10˜30 min to get disperse phase of granules (Angle of repose 39°). The granules obtained were homogeneous and had little powder, but had no effect on the follow-up process;
3) Mixed the granules obtained in step 2) and extragranular excipients for 10˜20 min to get total mixing granules;
4) Compressed the total mixing granules obtained in step 3) to prepare Clopidogrel Hydrogen Sulfate solid preparation (Strength: 75 mg).

No sticking occurred during compressing, and the surface of Clopidogrel Hydrogen Sulfate solid preparation obtained was smooth.

### COMPARISON EXAMPLE 1

| | |
|---|---|
| Item | Mass Ratio |

| | | |
|---|---|---|
| Intragranular | Clopidogrel Hydrogen Sulfate | 1 |
| Excipients | Microcrystalline cellulose | 0.05 |
| | PEG6000 | 0.2 |
| | Low substituted hydroxypropyl | 0.3 |
| | cellulose | |
| | Polyvinylpolypyrrolidone | 0.1 |
| Extragranular | Microcrystalline cellulose | 0.5 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

### Preparation Steps:

1) Mixed the prescription amount of Clopidogrel Hydrogen Sulfate (D90=116 μm) and excipients for 20˜30 min to get mixing powder;
2) Added the mixing powder obtained in step 1) into fluidized bed granulation machine, granulated at 50˜60° C. for 10˜30 min to get disperse phase of granules (Angle of repose 36°). The granules obtained were homogeneous and hardly had powder, but the granule diameter obtained was obviously larger than that obtained in example 1-5 by visual examination;
3) Mixed the granules obtained in step 2) and excipients for 10˜20 min to get total mixing granules;
4) Compressed the total mixing granules obtained in step 3) to prepare Clopidogrel Hydrogen Sulfate solid preparation (Strength: 75 mg).

No sticking occurred during compressing, and the surface of Clopidogrel Hydrogen Sulfate solid preparation obtained was smooth.

### COMPARISON EXAMPLE 2

| | |
|---|---|
| Item | Mass Ratio |

| | | |
|---|---|---|
| Intragranular | Clopidogrel Hydrogen Sulfate | 1 |
| Excipients | Microcrystalline cellulose | 0.05 |
| | PEG6000 | 0.2 |
| | Low substituted hydroxypropyl | 0.3 |
| | cellulose | |
| | Polyvinylpolypyrrolidone | 0.1 |
| Extragranular | Microcrystalline cellulose | 0.5 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

### Preparation Steps:

1) Mixed the prescription amount of Clopidogrel Hydrogen Sulfate (D90=23 μm) and intragranular excipients for 20˜30 min to get mixing powder;
2) Added the mixing powder obtained in step 1) into fluidized bed granulation machine, granulated at 50˜60° C. for 10˜30 min to get granules (Angle of repose 45°). The granulation effect was poor under this formula, and there was much powder in the granules obtained;
3) Mixed the granules obtained in step 2) and extragranular excipients for 10˜20 min to get total mixing granules;
4) Compress the total mixing granules obtained in step 3), and sticking occurred soon after compressing. The surface of Clopidogrel Hydrogen Sulfate solid preparation obtained was blurry, and the sticking still couldn't be avoided after adjustment of compressing process parameters repeatedly.

### COMPARISON EXAMPLE 3

Used the same batch of raw material to comparison example 2, and hoped to reach the purpose of optimizing granulation by increasing the usage amount of binder and adjusting the usage amount and adding way of other intragranular excipients, and to solve the sticking during compressing.

| | |
|---|---|
| Item | Mass Ratio |

| | | |
|---|---|---|
| Intragranular | Clopidogrel Hydrogen Sulfate | 1 |
| Excipients | Microcrystalline cellulose | 0.3 |
| | PEG6000 | 0.8 |
| | Low substituted hydroxypropyl | 0.4 |
| | cellulose | |
| | Polyvinylpolypyrrolidone | 0.35 |
| Extragranular | Sucrose stearate | 0.05 |
| Excipients | Magnesium stearate | 0.015 |

### Preparation Steps:

1) Mixed the prescription amount of Clopidogrel Hydrogen Sulfate (D90=23 μm) and intragranular excipients for 20˜30 min to get mixing powder;
2) Added the mixing powder obtained in step 1) into fluidized bed granulation machine, granulated at 50˜60° C. for 10˜30 min to get granules (Angle of repose 38°). The granules obtained were slightly smaller than that of example 1-5, but the granules were homogeneous and hardly have powder;
3) Mixed the disperse phase of granules obtained in step 2) and prescription amount of excipients for 10˜20 min to get total mixing granules;
4) Compressed the total mixing granules obtained in step 3) to prepare Clopidogrel Hydrogen Sulfate solid preparation (Strength: 75 mg).

No sticking occurred during compressing, and the surface of Clopidogrel Hydrogen Sulfate solid preparation obtained was smooth.

### EXAMPLE 6

Tested the dissolution of Clopidogrel Hydrogen Sulfate tablets obtained in examples 1-5 and comparisons example 1 and 3 using Method 2 paddle method, Appendix XC Dissolution Test Method in Chinese Pharmacopoeia 2010, and the results were as below:

| Item | 30 min Dissolution (%) |
|---|---|
| Example 1 | 97 |
| Example 2 | 95 |
| Example 3 | 97 |
| Example 4 | 95 |
| Example 5 | 95 |
| Comparison | 86 |
| example 1 | |
| Comparison | 98 |
| example 3 | |

Above all, dissolution of the tablets obtained in examples 1-5 could reach more than 90% within 30 min, and it complied with the requirements of clinical medication;

For comparison example 1, due to the raw material used had a larger granule diameter, the granule diameter of the disperse phase of granules obtained was larger, and the dissolution rate of the tablets was obviously slower than that in examples 1-5.

For comparison example 3, the dissolution was similar to the tablets obtained in examples 1-5, but the raw material used had a smaller granule diameter, the usage amount of binder and intragranular excipients should be increased to optimize granulation when preparing granules, which made the content of active ingredient in the disperse phase of granules low and granulation efficiency was far lower than those of examples 1-5.

### EXAMPLE 7

### Stability Test

Packaged the Clopidogrel Hydrogen Sulfate tablets obtained in examples 1-5, put it in high temperature and humidity condition (RH75%, 40° C.), and observed changes of the content of total impurities. The results were as below:

| Item | 0 day (%) | 15 day (%) | 30 day (%) |
|---|---|---|---|
| Example 1 | 0.3 | 0.3 | 0.4 |
| Example 2 | 0.3 | 0.4 | 0.4 |
| Example 3 | 0.3 | 0.4 | 0.4 |
| Example 4 | 0.3 | 0.3 | 0.4 |
| Example 5 | 0.3 | 0.4 | 0.4 |

Above all, preparations obtained in examples 1-5 showed higher stability during storage.

The execution examples above are the better mode of execution of this invention, but the mode of execution of this invention is not restricted by examples above. Any changes, ornament, replacement, combination and simplification without violation the spirit and principle of this invention shall be all equivalent substitute mode, and included in the protection range of this invention.

1. A Clopidogrel Hydrogen Sulfate solid preparation, includes granules and extragranular excipients, the granules include Clopidogrel Hydrogen Sulfate, filler, disintegrating agent and binder, the Clopidogrel Hydrogen Sulfate solid preparation uses melting granulation process, characterized by D90 of the Clopidogrel Hydrogen Sulfate is 30˜100 μm and binder is PEG6000, when the mass ratio of Clopidogrel Hydrogen Sulfate is 1, the usage amount of adhesion agent is 0.15˜0.35.

2. According to the Clopidogrel Hydrogen Sulfate solid preparation in claim 1, wherein D90 of Clopidogrel Hydrogen Sulfate is 40˜90 μm.

3. According to the Clopidogrel Hydrogen Sulfate solid preparation in claim 1, wherein D90 of Clopidogrel Hydrogen Sulfate is 50˜80 μm, and X-ray powder diffraction pattern of Clopidogrel Hydrogen Sulfate shows diffraction peaks while 2θ is 9.28°, 10.96°, 14.88°, 15.55°, 18.54°, 19.02°, 20.65°, 23.24°, 23.92° or 25.59°.

4. According to the Clopidogrel Hydrogen Sulfate solid preparation in claim 1, wherein the filler of granules is one or mixture in various amounts of more than two of microcrystalline cellulose, Lactose and pregelatinized starch, and the disintegrating agent of granules is one or mixture in various amounts of more than two of polyvinylpolypyrrolidone, low-substituted hydroxypropyl cellulose, croscarmellose sodium and sodium carboxymethyl starch, when the mass ratio of Clopidogrel Hydrogen Sulfate is 1, the mass ratio of disintegrating agent is 0.2˜0.5, mass ratio of the filler is 0.03˜0.1.

5. According to the Clopidogrel Hydrogen Sulfate solid preparation in claim 1, wherein the formula of disperse phase granules is any one of the following:
Formula 1

| Item | Mass Ratio |
|---|---|
| Clopidogrel Hydrogen Sulfate | 1 |
| Microcrystalline cellulose | 0.05 |
| PEG6000 | 0.2 |
| Low substituted hydroxypropyl | 0.3 |
| cellulose | |
| Polyvinylpolypyrrolidone | 0.1 |

Formula 2

| Item | Mass Ratio |
|---|---|
| Clopidogrel Hydrogen Sulfate | 1 |
| Lactose | 0.06 |
| PEG6000 | 0.3 |
| Croscarmellose sodium | 0.3 |

Formula 3

| Item | Mass Ratio |
|---|---|
| Clopidogrel Hydrogen Sulfate | 1 |
| Microcrystalline cellulose | 0.05 |
| PEG6000 | 0.2 |
| Polyvinylpolypyrrolidone | 0.4 |

Formula 4

| Item | Mass Ratio |
|---|---|
| Clopidogrel Hydrogen Sulfate | 1 |
| Microcrystalline cellulose | 0.05 |
| PEG6000 | 0.15 |
| Low substituted hydroxypropyl | 0.3 |
| cellulose | |
| Polyvinylpolypyrrolidone | 0.1 |

Formula 5

| Item | Mass Ratio |
|---|---|
| Clopidogrel Hydrogen Sulfate | 1 |
| Lactose | 0.1 |
| PEG6000 | 0.35 |
| Croscarmellose sodium | 0.3 |

6. According to the Clopidogrel Hydrogen Sulfate solid preparation in claim 1, wherein extragranular excipients include filler, disintegrating agent, stabilizer and lubricant, the filler is one or mixture in various amounts of more than two of microcrystalline cellulose, Lactose and pregelatinized starch; The disintegrating agent is one or mixture in various amounts of more than two of polyvinylpolypyrrolidone, hydroxypropyl cellulose, low-substituted hydroxypropyl cellulose, croscarmellose sodium and sodium carboxymethyl starch; stabilizer is sucrose stearate; and lubricant is one or mixture in various amounts of more than two of magnesium stearate, hydrogenated castor oil and talcum powder, when the mass ratio of Clopidogrel Hydrogen Sulfate is 1, the mass ratio of filler is 0.3˜1.0, the mass ratio of disintegrating agent is 0.1˜0.5, the mass ratio of stabilizer is 0.02˜0.08, and the mass ratio of lubricant is 0.03˜1.0.

7. According to the Clopidogrel Hydrogen Sulfate solid preparation in claim 1, wherein the formula of disperse phase granules is any one of the following:
Formula 1

| | |
|---|---|
| Item | Mass Ratio |
| | | |
|---|---|---|
| Intragranular | Clopidogrel Hydrogen Sulfate | 1 |
| Excipients | Microcrystalline cellulose | 0.05 |
| | PEG6000 | 0.2 |
| | Low substituted hydroxypropyl | 0.3 |
| | cellulose | |
| | Polyvinylpolypyrrolidone | 0.1 |
| Extragranular | Microcrystalline cellulose | 0.5 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

Formula 2

| | |
|---|---|
| Item | Mass Ratio |
| | | |
|---|---|---|
| Intragranular | Clopidogrel Hydrogen Sulfate | 1 |
| Excipients | Lactose | 0.06 |
| | PEG6000 | 0.3 |
| | Croscarmellose sodium | 0.3 |
| Extragranular | Microcrystalline cellulose | 0.5 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

Formula 3

| | |
|---|---|
| Item | Mass Ratio |
| | | |
|---|---|---|
| Intragranular | Clopidogrel Hydrogen Sulfate | 1 |
| Excipients | Microcrystalline cellulose | 0.05 |
| | PEG6000 | 0.2 |
| | Polyvinylpolypyrrolidone | 0.4 |
| Extragranular | Microcrystalline cellulose | 0.5 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

Formula 4

| | |
|---|---|
| Item | Mass Ratio |
| | | |
|---|---|---|
| Intragranular | Clopidogrel Hydrogen Sulfate | 1 |
| Excipients | Microcrystalline cellulose | 0.05 |
| | PEG6000 | 0.15 |
| | Low substituted hydroxypropyl | 0.3 |
| | cellulose | |
| | Polyvinylpolypyrrolidone | 0.1 |
| Extragranular | Microcrystalline cellulose | 0.55 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

Formula 5

| | |
|---|---|
| Item | Mass Ratio |
| | | |
|---|---|---|
| Intragranular | Clopidogrel Hydrogen Sulfate | 1 |
| Excipients | Lactose | 0.1 |
| | PEG6000 | 0.35 |
| | Croscarmellose sodium | 0.3 |
| Extragranular | Microcrystalline cellulose | 0.5 |
| Excipients | Low substituted hydroxypropyl | 0.1 |
| | cellulose | |
| | Polyvinylpolypyrrolidone | 0.25 |
| | Sucrose stearate | 0.05 |
| | Magnesium stearate | 0.015 |

8. The preparation process of Clopidogrel Hydrogen Sulfate solid preparation in claim 1 includes the following steps:
1) Mix prescription amount of Clopidogrel Hydrogen Sulfate and intragranular excipients for 20˜30 min and get mixing powder;
2) Add mixing powder obtained in step 1) into melting granulation machine, and granulate at 50˜80° C. for 10˜30 min to get granules.

9. According to the preparation process of Clopidogrel Hydrogen Sulfate solid preparation in claim 8, it includes the following steps:
1) Mix prescription amount of Clopidogrel Hydrogen Sulfate and intragranular excipients for 20˜30 min and get mixing powder;
2) Add disperse phase of mixing powder obtained in step 1) into melting granulation machine, and granulate at 50˜80° C. for 10˜30 min to get disperse phase granules;
3) Mix the disperse phase granules obtained in step 2) and prescription amount of extragranular excipients for 10˜20 min, and get total mixing granules;
4) Compress the total mixing granules obtained in step 3) to prepare Clopidogrel Hydrogen Sulfate solid preparation.

10. According to the preparation process of Clopidogrel Hydrogen Sulfate solid preparation in claim 9, wherein in steps 1) and 3), column lifting blender is used; the melting granulation machine in step 2) is jacketed kettle or fluidized bed granulation machine; the compressing pressure in 4) is 3˜5 kgf, and the preparation method includes coating step.
